(19) United States
(12) Patent Application Publication
(10) Pub. No.: US 2020/0013033 A1
(43) Pub. Date: Jan. 9, 2020

(54) USE FEE CALCULATION DEVICE FOR MEDICAL INSTRUMENT MANAGEMENT DEVICE AND MEDICAL INSTRUMENT MANAGEMENT SYSTEM

(71) Applicant: DGSHAPE Corporation, Hamamatsu-shi (JP)

(72) Inventor: Masaki HANAJIMA, Hamamatsu-shi (JP)

(21) Appl. No.: 16/455,831

(22) Filed: Jun. 28, 2019

(30) Foreign Application Priority Data
Jul. 4, 2018 (JP) 2018-127400

(51) Int. Cl. G06Q 20/14; G16H 40/40; G16H 40/60

(52) U.S. Cl. CPC G06Q 20/145; G16H 40/60; G16H 40/40

(57) ABSTRACT

A use fee calculation device includes a receiver that is connected to at least one reading device and receives read information when an identification label of a medical instrument has been read by the reader and user information in association with one another, a counter that counts, based on the read information and the use information that have been received by the receiver, a number of times of use, which is a number of pieces of the read information that have been received by the receiver, for each of the users, and a fee calculator that calculates, based on the number of times of use for each of the users, which has been counted by the counter, and a unit price that has been set in advance, a use fee for each of the users.

### CROSS REFERENCE TO RELATED APPLICATIONS

This application claims the benefit of priority to Japanese Patent Application No. 2018-127400 filed on Jul. 4, 2018. The entire contents of this application are hereby incorporated herein by reference.

### BACKGROUND OF THE INVENTION

### 1. Field of the Invention

The present invention relates to a use fee calculation device for a medical instrument management device and a medical instrument management system including the medical instrument management device and the use fee calculation device.

### 2. Description of the Related Art

For example, as a system related to a medical instrument that is used in a medical practice, such as a surgical operation, a medical examination, or the like, a medical practice management system that manages a schedule and performance contents of a medical practice is disclosed in Japanese Patent No. 4873384. In the medical practice management system, a medical practitioner (for example, a doctor or a nurse) inputs a surgical operation schedule (which will be hereinafter referred to as “schedule information”) by selecting at least one surgical method from a plurality of surgical methods that have been set in advance. Also, in the medical practice management system, data in which information of a medical instrument that is used in a medical practice and a surgical method are associated with one another is stored. In accordance with a surgical method that has been selected by the medical practitioner, information of a medical instrument is selected.

An identification number is given to the medical instrument. When the medical practitioner registers performance contents of a surgical operation, the medical practitioner reads the identification number of the medical instrument that is used in the surgical operation using a reading device. Then, the identification number that has been read by the reading device is managed in association with the above described schedule information in the medical practice management system. Thus, the schedule information of the surgical operation and the performance contents, such as the medical instrument that is used in the surgical operation or the like, are associated with one another. Therefore, as data of data included in the performance contents, which is included in the schedule information, data of the above described schedule information that has been input can be used. Thus, management of the performance contents can be facilitated. A user of the medical practice management system concludes a license agreement with a provider and pays a predetermined use fee to the provider.

Incidentally, depending on medical instruments, some medical instruments are washed or sterilized after being used and thus can be repeatedly used. In such a medical instrument that is repeatedly used, a plurality of predetermined processes is sequentially performed on the medical instrument. In a medical institution, such as a hospital or the like, in which there are many medical instruments that are repeatedly used, management of the medical instruments is complicated, and therefore, in some cases, a medical instrument management device used for managing the medical instruments is introduced from a provider. In such a case, the user can use the medical instrument management device by paying a predetermined use fee to the provider.

Incidentally, depending on users, the number of medical instruments owned by a user is different from that of another user, and the number of medical instruments that are subjects of a medical instrument management device differs. Therefore, depending on users, a use amount, such as the number of times of use, use time, or the like, of a medical instrument management device differs. For example, in a case in which a use fee is fixed for all of users, users the use amount of which is relatively small feel that the use fee is expensive. Also, in other cases, a provider provides a medical instrument management device to a plurality of users. It is preferable for providers that a work time related to a use fee of users is short.

### SUMMARY OF THE INVENTION

In view of the foregoing, preferred embodiments of the present invention provide use fee calculation devices for medical instrument management devices and medical instrument management systems that each calculate a proper use fee for a user who uses the medical instrument management device and are able to reduce a work time of a provider of the medical instrument management device, which is related to the use fee.

A use fee calculation device for a medical instrument management device according to a preferred embodiment of the present invention includes a receiver, a counter, and a fee calculator. The receiver is a receiver that is directly or indirectly connected to at least one reading device and receives read information when an identification label provided in a medical instrument has been read by the reader and user information that is recorded in association with the read information and specifies a user who uses the reading device. The counter counts, based on the read information and the user information that have been received by the receiver, the number of times of use, which is the number of pieces of the read information that have been received by the receiver, for each of the users. The fee calculator calculates, based on the number of times of use for each of the users, which has been counted by the counter, and a unit price that has been set in advance, a use fee for each of the users.

With the use fee calculation device for a medical instrument management device according to a preferred embodiment of the present invention, read information when a user has read an identification label of a medical instrument using the reading device and user information are received by the receiver. Therefore, the number of times of use can be obtained by counting, based on the read information and the user information, the number of pieces of read information for each user. Then, based on the number of times of use and a unit price, a use fee in accordance with the number-of-times-of-use for the user can be calculated. Accordingly, for a user the number of times of use of which is small, a relatively low use fee can be charged and, for a user the number of times of use of which is large, a relatively high use fee can be charged. Thus, a use fee in accordance with the number of times of use of a user can be charged to the user. Therefore, a proper use fee for the user can be calculated. Also, in at least one preferred embodiment of the present invention, at least one reading device is connected to the use fee calculation device. Therefore, for example, even in a case in which the reading device is installed in a remote location, the read information and the user information are able to be put together in the use fee calculation device. Accordingly, the use fee for each user is able to be calculated in a place in which the read information and the user information have been received. Thus, the use fee is able to be calculated without a visit of a provider to a place in which the reading device is installed. Therefore, an operation time related to the use fee is able to be reduced.

According to this preferred embodiment, a proper use fee for a user who uses the medical instrument management device is able to be calculated and an operation time related to a use fee for a provider of the medical instrument management device is able to be reduced.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a conceptual diagram illustrating a relationship between a medical instrument management system and a circulation cycle according to a first preferred embodiment of the present invention.

FIG. 2 is a conceptual diagram illustrating a medical instrument management system.

FIG. 3 is a block diagram of a medical instrument management device and a use fee calculation device.

FIG. 4 is a table illustrating an example of a configuration of a use history table.

FIG. 5 is a table illustrating an example of a configuration of a number-of-times-of-use table.

FIG. 6 is a table illustrating an example of a configuration of a use fee table.

FIG. 7 is a flowchart illustrating an example of procedures for calculating a use fee.

FIG. 8 is a block diagram of a medical instrument management system according to a second preferred embodiment of the present invention.

FIG. 9 is a table illustrating an example of a configuration of a manual use history table.

FIG. 10 is a table illustrating an example of a configuration of a number-of-times-of-manual-use table.

FIG. 11 is a table illustrating an example of a configuration of a manual use fee table.

FIG. 12 is a flowchart illustrating an example of procedures for calculating a manual use fee.

### DETAILED DESCRIPTION OF THE PREFERRED EMBODIMENTS

With reference to the attached drawings, medical instrument management systems each including a use fee calculation device for a medical instrument management device (which will be hereinafter referred to merely as a “use fee calculation device”) according to preferred embodiments of the present invention will be described below. Note that, as a matter of course, preferred embodiments described herein are not intended to be particularly limiting the present invention. Also, members and components that have the same function are denoted by the same reference character and redundant description will be omitted or simplified, as appropriate.

### First Preferred Embodiment

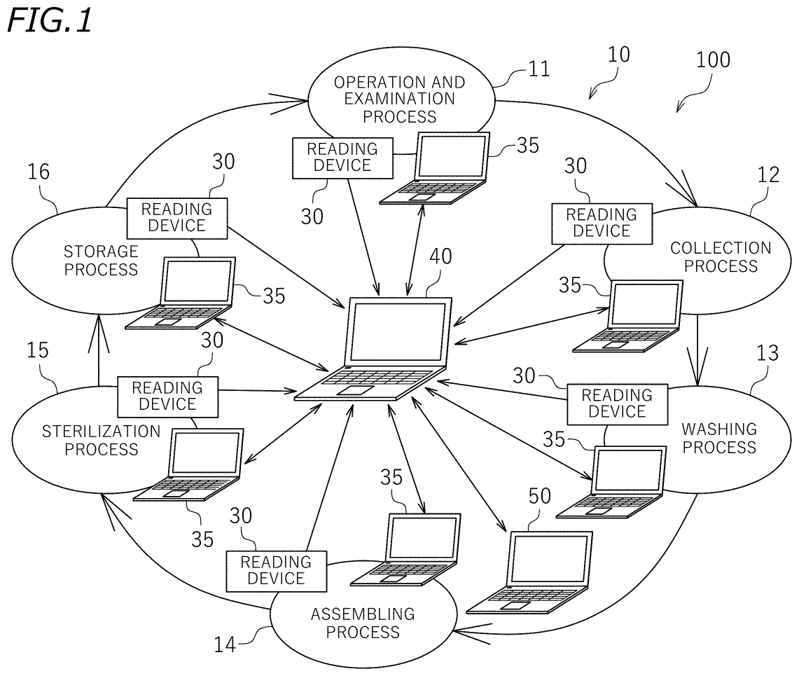

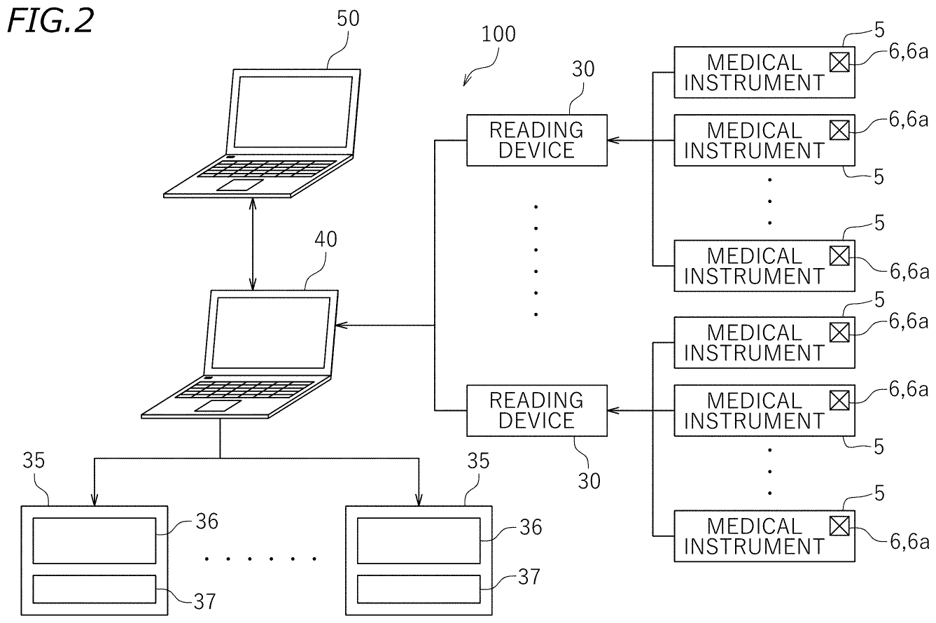

FIG. 1 is a conceptual diagram illustrating a relationship between a medical instrument management system 100 and a circulation cycle 10 according to a first preferred embodiment of the present invention. FIG. 2 is a conceptual diagram illustrating the medical instrument management system 100. As illustrated in FIG. 2, the medical instrument management system 100 is a system that manages a medical instrument 5 that is used by a user. As used herein, a term “user” means not only a person but also an organization, such as an enterprise, a group, a hospital, or the like, a section of the organization, a process, or the like.

The medical instrument 5 is an instrument that is used in a medical practice that is performed in a hospital. The medical instrument 5 is, for example, a trocar, forceps, an incision device, a washing and sucking device, scissors, a scalpel (for example, a laser scalpel or an electric scalpel), an ultrasonic cutter, a scalpel holder, a cannula, tweezers, a retractor, a scale, a sonde, an elevator, a rasp, a suction tube, a rib retractor, a rib contractor, a needle holder, an injector, a metal ball, a pus tray, a cup, a pin, a mirror, a file, an opening tool, a clamp, a handpiece, an elevatorium, a chisel, a sharp spoon, a raspatory, a speculum, a suture needle, a punch, a water receiver, a needle, a penetrator, a bougie, a vent pipe, a bone impactor, a luer, a radio plier, a hammer, an angle gauge, a thermometer, a perforator, a spuit, a metal swab, an enema, a syringe, an endoscope, or the like. However, the above described examples are merely some of examples of the medical instrument 5 and the medical instrument 5 is not limited to the above described examples.

The medical instrument 5 may be formed of a single component and may be formed of a plurality of components. Examples of the medical instrument 5 formed of a plurality of components include, for example, a trocar, forceps, an incision device, a washing and sucking device, or the like which is used in a laparoscopic surgical operation. The medical instrument 5 described above is formed of a plurality of components, is collected after a surgical operation, and, thereafter, is disassembled to a plurality of components. Elements or parts that form the medical instrument 5 are referred to as components herein. Also, in some cases, such a component is further formed of a plurality of components, and in such a case, all of the components are referred to as components.

As illustrated in FIG. 1, the medical instrument 5 is repeatedly usable in a predetermined circulation cycle 10. Note that the number of repetitions of the medical instrument 5 in the circulation cycle 10 is not particularly limited. The number of repetitions differs depending on a type of the medical instrument or a component forming the medical instrument 5. In this preferred embodiment, the circulation cycle 10 includes an operation and examination process 11, a collection process 12, a washing process 13, an assembling process 14, a sterilization process 15, and a storage process 16.

The operation and examination process 11 is a process in which a surgical operation or a medical examination is performed using the medical instrument 5. In the operation and examination process 11, the medical instrument 5 that is stored in a predetermined place is taken out from the predetermined place and a surgical operation or a medical examination is performed using the medical instrument 5. The collection process 12 is a process that is performed after the operation and examination process 11. The collection process 12 is a process in which, after a surgical operation or a medical examination is performed, the medical instrument 5 that has been used therein is collected. Note that, in a case in which the medical instrument 5 is formed of a plurality of components, the collection process 12 may include an operation of disassembling the medical instrument 5 to individual components. The washing process 13 is a process that is performed after the collection process 12. The washing process 13 is a process in which the collected medical instrument 5 or each of the collected individual components of the medical instrument 5 is washed. In the washing process 13, for example, using a washer (not illustrated), washing of the medical instrument 5 is performed.

The assembling process 14 is a process that is performed after the washing process 13. The assembling process 14 is a process in which the components of the medical instrument 5 which have been washed are assembled to complete the single medical instrument 5. The assembling process 14 is a process in which the medical instrument 5 that is formed of a plurality of components is assembled. In this preferred embodiment, the assembling process 14 includes sorting a plurality of medical instruments 5 as a set. As used herein, “sorting as a set” is, for example, putting the plurality of medical instruments 5 into an arbitrary case and thus collectively storing the medical instruments 5. The sterilization process 15 is a process that is performed after the assembling process 14. The sterilization process 15 is a process in which the medical instrument 5 is sterilized. In the sterilization process 15, for example, using a sterilizer (not illustrated), sterilization processing of the medical instrument 5 is performed. Sterilization processing will be hereinafter also referred to merely as sterilization. The storage process 16 is a process that is performed after the sterilization process 15. The storage process 16 is a process in which the medical instrument 5 that has been sterilized is stored in the predetermined place. Note that, after the storage process 16, the operation and examination process 11 is performed. In this preferred embodiment, the processes 11 to 16 are examples of a predetermined process of a preferred embodiment of the present invention. For example, the collection process 12 is an example of a first process of a preferred embodiment of the present invention. The washing process 13 is an example of a second process of a preferred embodiment of the present invention.

In this preferred embodiment, as illustrated in FIG. 2, an identification label 6 is given to the medical instrument 5. The identification label 6 is used to distinguish one medical instrument 5 from another medical instrument 5. For example, an instrument ID 6a is recorded in the identification label 6. The instrument ID 6a is a character string including a number, an alphabet, or the like. The instrument ID 6a differs for each medical instrument 5. For example, the instrument ID 6a is given for each medical instrument 5. However, in a case in which the medical instrument 5 is formed of a plurality of components and is disassembled to the plurality of components in the collection process 12, the identification label 6 is provided for each of the components and the instrument ID 6a is given for each of the components. Note that there is no particular limitation on a type of the identification label 6. For example, the identification label 6 may be a one-dimensional bar code, may be a two-dimensional bar code, and may be an IC tag. In this preferred embodiment, the instrument ID 6a of the medical instrument 5 can be acquired by reading the identification label 6 using a reading device 30, which will be described later.

In this preferred embodiment, the medical instrument management system 100 is a system that manages the medical instrument 5 in each of the processes 11 to 16. In this case, the medical instrument management system 100 performs management about which one of operations of the processes 11 to 16 is performed for the medical instrument 5 by reading the instrument ID 6a from the identification label 6 given to the medical instrument 5 in each of the processes 11 to 16. Also, the medical instrument management system 100 may be a system that performs management of inventory of the medical instrument 5.

In this preferred embodiment, as illustrated in FIG. 1, the medical instrument management system 100 preferably includes at least one reading device 30, at least one terminal 35, a medical instrument management device 40, and a use fee calculation device 50.

As illustrated in FIG. 2, the reading device 30 reads the instrument ID 6a of the identification label 6 given to the medical instrument 5. The reading device 30 reads the instrument ID 6a recorded in the identification label 6 and transmits the instrument ID 6a that has been read by the reading device 30 to the medical instrument management device 40. Note that there is no particular limitation on a type of the reading device 30 and the reading device 30 may be a device that can read the instrument ID 6a of the identification label 6. For example, in a case in which the identification label 6 is a two-dimensional code, the reading device 30 may be a two-dimensional code reader that can read a two-dimensional code. For example, in a case in which the identification label 6 is an IC tag, the reading device 30 may be an IC tag reader. In this preferred embodiment, the reading device 30 is a non-contact device. However, the reading device 30 may be a contact device. Note that, as illustrated in FIG. 1, the reading device 30 is provided in each of the processes 11 to 16. An installation site of the reading device 30 is, for example, in an operation space in which each of the processes 11 to 16 is performed. However, the reading device 30 may not be provided in some of the processes 11 to 16. Note that management of the medical instrument 5 in a process in which the reading device 30 is not provided is not performed in the medical instrument management system 100. In each of the processes 11 to 16, an operator reads the identification label 6 given to the medical instrument 5 using the reading device 30 before the corresponding operation is performed.

The terminal 35 is provided for each of the processes 11 to 16. For example, the terminal 35 is paired with the reading device 30 and thus is provided in each of the processes 11 to 16. The terminal 35 is provided for each of the processes 11 to 16 for which the reading device 30 is installed. The terminal 35 may be, for example, a tablet terminal and may be a desktop or notebook computer. As illustrated in FIG. 2, the terminal 35 includes, for example, a display screen 36 and an operation section 37, such as a keyboard, a mouse, or the like.

The medical instrument management device 40 is a device that manages inventory of the medical instrument 5 or the like. Also, the medical instrument management device 40 is a device that manages which one of the operations of the processes 11 to 16 is performed on the medical instrument 5. In this case, management of the medical instrument 5 includes at least the above described two managements. Note that description of a specific configuration of the medical instrument management device 40 or the like will be omitted as appropriate. In this preferred embodiment, the instrument ID 6a of the identification label 6 is read by the reading device 30, and the medical instrument 5 when the instrument ID 6a is transmitted to the medical instrument management device 40 is a subject to be managed. For example, the medical instrument management device 40 is a computer and includes a CPU, a ROM in which a program that is executed by the CPU is stored, a RAM, or the like. In this case, the medical instrument 5 is managed using a program that is stored in the computer.

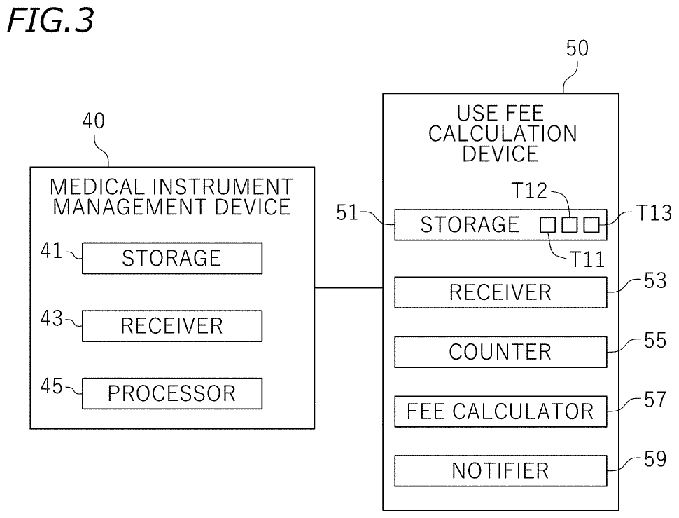

FIG. 3 is a block diagram of the medical instrument management device 40 and a use fee calculation device 50. In this preferred embodiment, as illustrated in FIG. 3, the medical instrument management device 40 includes a storage 41 in which a predetermined table of a database is stored, a receiver 43 that receives the instrument ID 6a that has been transmitted from the reading device 30, and a processor 45 that performs processing of managing the medical instrument 5 with the given instrument ID 6a, which has been received by the receiver 43. The processor 45 causes at least the instrument ID 6a, the process ID, and reception date and time to be stored in association with one another, for example, in the table stored in the storage 41. Thus, which one of the operations of the processes 11 to 16 is performed on the medical instrument 5 that corresponds to the instrument ID 6a is able to be managed. Also, for the medical instrument 5 that corresponds to the instrument ID 6a, the processor 45 may be configured or programmed to cause the display screen 36 (see FIG. 2) of the terminal 35 of the process to display contents of an operation instruction that indicates what kind of operation is performed in a process associated with the instrument ID 6a thereon. Thus, the operator of the process can properly perform an operation in accordance with the contents of the operation instruction that is displayed on the display screen 36 of the terminal 35.

Incidentally, a provider of the medical instrument management device 40 of the medical instrument management system 100 charges a predetermined use fee to a user who introduces the medical instrument management device 40. In this case, users who have introduced the above described medical instrument management device 40 include a user who has introduced at least a portion of the medical instrument management system 100. For example, a user who uses only the reading device 30 accesses the medical instrument management device 40 via the reading device 30, and therefore, is included in the users who have introduced the medical instrument management device 40.

Depending on users, the number of medical instruments 5 owned by a user is different from that of another user, and the number of medical instruments 5 that are management subjects of the medical instrument management device 40 differs. Also, depending on users, there are some users who want the medical instrument management device 40 to perform management of the medical instrument 5 for all of the processes 11 to 16 and other users who want the medical instrument management device 40 to perform management of the medical instrument 5 for some of the processes 11 to 16. Therefore, depending on users, an amount of use, such as the number of times of use, use time, or the like, of the medical instrument management device 40 differs. For example, in a case in which a fixed use fee is charged to all of users who have introduced the medical instrument management device 40, users the amount of use of which is relatively small feel that the use fee is expensive. Therefore, it is preferable to charge a proper use fee for each of all of users. To solve this problem, in this preferred embodiment, a meter-rate charging system in which a use fee is set for each user in accordance with the amount of use is preferably used.

In the above described meter-rate charging system, for example, an operation of counting the number of times the identification label 6 of the medical instrument 5 has been read by the reading device 30 for each user is needed. The number of times the identification label 6 has been read by the reading device 30 will be hereinafter referred to as the number of times of use. For example, in a case in which the medical instrument management system 100 uses a so-called stand-alone system, a provider has to visit an introduction destination of a user and count the number of times of use. Therefore, there is a possibility that an operation time related to a use fee for each user is relatively increased. To solve this problem, this preferred embodiment uses a so-called client-server system for the medical instrument management system 100.

The use fee calculation device 50 according to this preferred embodiment, a device that can solve the above described two problems. Next, the use fee calculation device 50 will be described. The use fee calculation device 50 is a device that calculates a use fee in accordance with the number of times of use of the medical instrument management system 100 for a user who uses the medical instrument management system 100 including the medical instrument management device 40. In this case, the use fee calculation device 50 sets a use fee in accordance with the number of times the user has read the identification label 6 of the medical instrument 5 using the reading device 30. The medical instrument management system 100 according to this preferred embodiment does not use a fixed rate charging system but uses a meter-rate charging system.

The use fee calculation device 50 is a computer and includes a CPU, a ROM in which a program that is executed by the CPU is stored, a RAM, or the like. In this case, a use fee of the medical instrument management system 100 is calculated for each user using a program stored in the computer. The use fee calculation device 50 may be realized by a dedicated computer and may be realized by a general-purpose computer.

In this preferred embodiment, the use fee calculation device 50 is electrically connected to at least one medical instrument management device 40 and is communicably connected thereto. In an example of FIG. 3, the use fee calculation device 50 is communicably connected to one medical instrument management device 40 for convenience of description. However, the use fee calculation device 50 may be communicably connected to a plurality of medical instrument management devices 40. The use fee calculation device 50 may be connected to at least one medical instrument management device 40 via a wired communication and may be connected thereto via a wireless communication. In this case, the medical instrument management system 100 is a so-called client-server system. In the medical instrument management system 100 according to this preferred embodiment, for example, the medical instrument management device 40 and the use fee calculation device correspond to a server and the reading device 30 and the terminal 35 correspond to a client. However, the medical instrument management system 100 may be a so-called cloud system. The use fee calculation device 50 may be provided integrally with the medical instrument management device 40 and be realized together with the medical instrument management device 40 by one computer. The use fee calculation device 50 may be a separate device from the medical instrument management device 40 and be realized by a different computer from a computer of the medical instrument management device 40.

In this preferred embodiment, as illustrated in FIG. 3, the use fee calculation device 50 includes the storage 51, a receiver 53, a counter 55, a fee calculator 57, and a notifier 59.

Each of the above described components may be configured of a software and may be configured of a hardware. Also, each of the above described components may be realized by one or more processors and may be incorporated in a circuit.

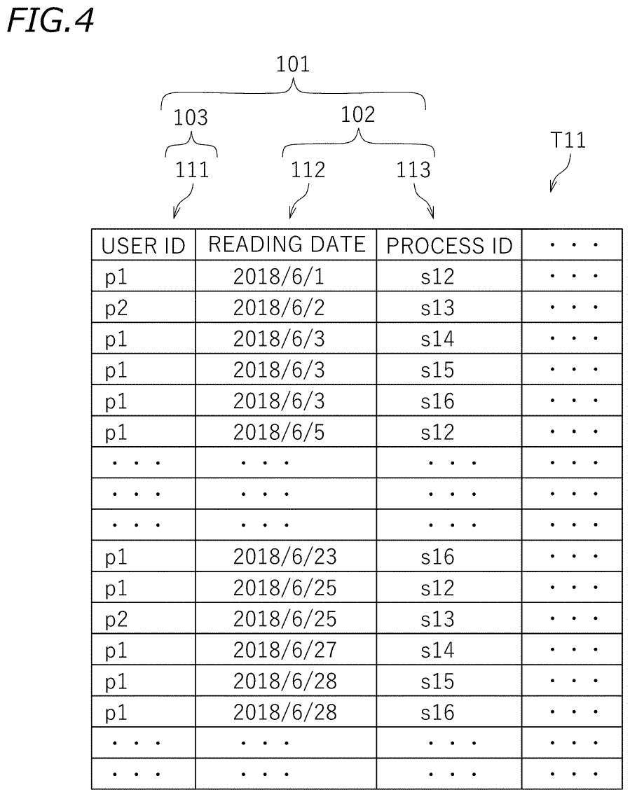

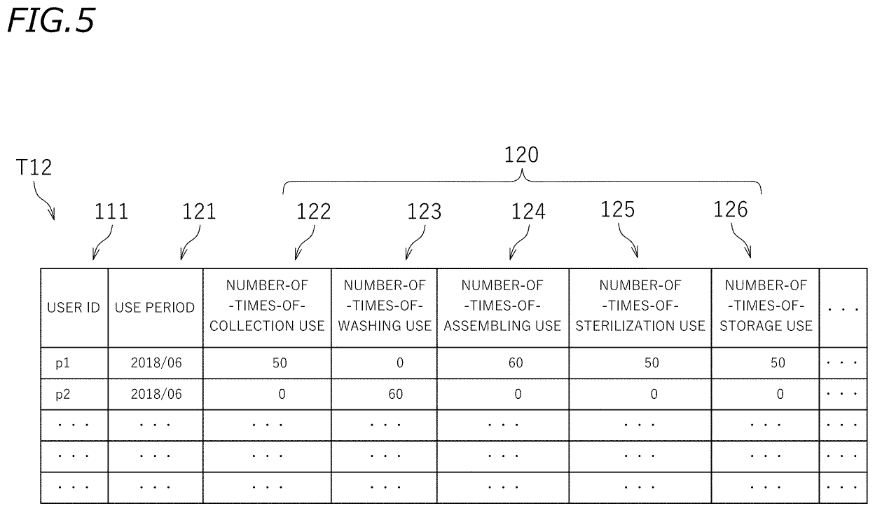

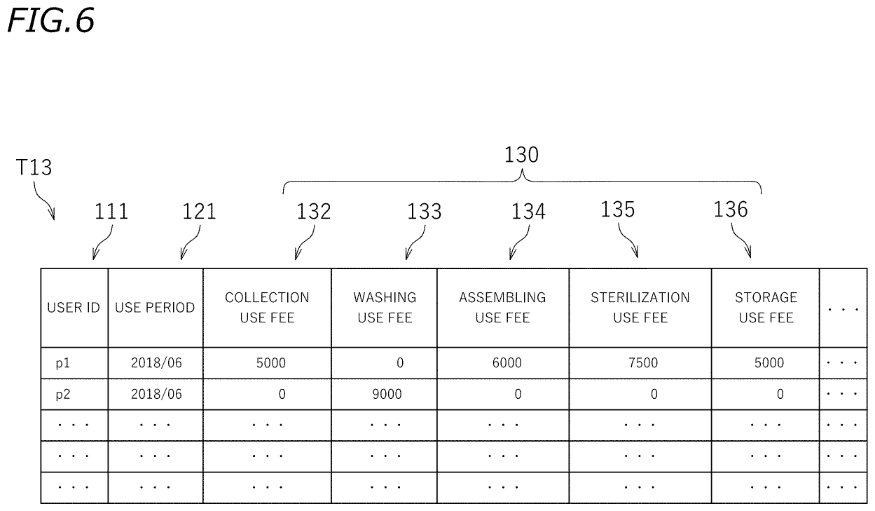

FIG. 4 is a table illustrating an example of a configuration of a use history table T11. FIG. 5 is a table illustrating an example of a configuration of a number-of-times-of-use table T12. FIG. 6 is a table illustrating an example of a configuration of a use fee table T13. In the storage 51, the use history table T11 (see FIG. 4), the number-of-times-of-use table T12 (see FIG. 5), and the use fee table T13 (see FIG. 6) are stored in advance. As used herein, a table is a table that forms a database.

The receiver 53 is indirectly connected to at least one reading device 30 via the medical instrument management device 40. The receiver 53 receives reception information 101 (specifically, read information 102 and user information 103) when the identification label 6 given to the medical instrument 5 is read by the reading device 30. In a predetermined process (each of the processes 11 to 16) in which a predetermined operation is performed on the medical instrument 5, the identification label 6 that has been given to the medical instrument 5 is read before the operation is performed, and thus, the receiver 53 receives the reception information 101. In this case, the reception information 101 is information that is sent to the use fee calculation device 50 when the identification label 6 of the medical instrument 5 is read by the reading device 30 and is information that is used when calculation of a use fee is performed. In this preferred embodiment, as illustrated in FIG. 4, the reception information 101 includes the read information 102 and the user information 103.

The read information 102 is information when the user reads the identification label 6 given to the medical instrument 5 using the reading device 30. The read information 102 includes a reading date 112 that is, for example, a date on which the reading device 30 has read the identification label 6 and the process ID 113 that corresponds to a process in which an operation is performed in an operation space in which the reading device 30 is installed. In this case, the reading date 112 and the process ID 113 are associated with one another. Note that the reading date 112 may include, in addition to the date, a time at which the identification label 6 of the medical instrument 5 has been read. Note that, in this preferred embodiment, the process ID 113 is an example of process information according to a preferred embodiment of the present invention.

In this preferred embodiment, the reading device 30 is arranged in the space in which the operation of each of the processes 11 to 16 is performed. That is, for each of the processes 11 to 16, the dedicated reading device 30 of a corresponding one of the processes 11 to 16 is provided. For example, the process ID 113 that indicates for which one of the processes 11 to 16 the reading device is dedicated may be assigned to the reading device 30. The reading device 30 transmits, when the instrument ID 6a of the medical instrument 5 is read by the reading device 30, the process ID 113 with the instrument ID 6a to the medical instrument management device 40. At this time, the medical instrument management device 40 acquires a reception date when the instrument ID 6a has been received and sets the reception date as the reading date 112. Then, the medical instrument management device 40 transmits the reading date 112 and the process ID 113 to the receiver 53 of the use fee calculation device 50. The receiver 53 that has received the reading date 112 and the process ID 113 causes the storage 51 to store the reading date 112 and the process ID 113 that have been received in association with one another.

Note that the process ID 113 is a unique ID that has been given to each of the processes 11 to 16 and, based on the process ID 113, which one of the processes 11 to 16 is performed can be specified. Although illustration is omitted, for example, a process table in which the process ID 113 and a process name are associated with one another is stored in the storage 51. The process name can be specified based on the process ID 113 by referring to the process table. Note that, in the read information 102, the process ID 113 may be replaced with the process name.

The user information 103 is information that specifies a user who has used the reading device 30 when the identification label 6 given to the medical instrument 5 is read using the reading device 30. The user information 103 is information recorded in association with the read information 102. For example, a user ID 111 is included in the user information 103. A user name or some other information related to the user may be included in the user information 103. In this preferred embodiment, the reading device 30 is a dedicated device of a specific user. For example, the user ID 111 that indicates which user's reading device the reading device 30 is may be assigned to the reading device 30. For example, in a case in which an ID is assigned to the reading device 30 itself, the ID and the user ID 111 are associated with one another. In this case, the user ID 111 may be replaced with the ID of the reading device 30 itself. When the instrument ID 6a of the medical instrument 5 is read by the reading device 30, the reading device 30 transmits the user ID 111 with the instrument ID 6a and the process ID 113 to the medical instrument management device 40. Then, the medical instrument management device 40 transmits the user ID 111 with the reading date 112 and the process ID 113 to the receiver 53 of the use fee calculation device 50. The receiver 53 causes the storage 51 to store the user ID 111, the reading date 112, and the process ID 113 in association with one another.

Note that the user ID 111 is a unique ID that has been given to each user and, based on the user ID 111, which user uses the reading device 30 can be specified. Although illustration is omitted, for example, in the storage 51, a user table in which the user ID 111 and the user name are associated with one another is stored. The user name can be specified based on the user ID 111 by referring to the user table. Note that, in the user information 103, the user ID 111 may be replaced with the user name.

In this preferred embodiment, when the reception information 101 is received by the receiver 53, the reception information 101 is stored in the use history table T11 illustrated in FIG. 4. The use history table T11 is a table in which, in each of the processes 11 to 16, a history of use of the medical instrument management system 100 by the user is recorded. The use history table T11 is stored in the storage 51 in advance. In this preferred embodiment, in the use history table T11, items of the user ID 111, the reading date 112, and the process ID 113 are stored in association with one another. Note that, in the use history table T11, some other item than the above described three items, that is, for example, the user name or the instrument ID 6a (see FIG. 2), may be included.

The counter 55 counts, based on the read information 102 and the user information 103 that have been received by the receiver 53, the number of times of use, which is the number of pieces of the read information 102 that have been received by the receiver 53 for each user. In this case, the counter 55 counts the number of times of use, that is, the number of times the receiver 53 has received the reception information 101, for each user, based on the read information 102 and the user information 103 of the reception information 101 that has been received by the receiver 53. In this preferred embodiment, the counter 55 counts the number of times of use for each user, based on the use history table T11 stored in the storage 51. Specifically, the counter 55 counts rows in which a specific user ID 111 is included in the use history table T11. Then, the counter 55 sets a count number of the counting as the number-of-times-of-use 120 (see FIG. 5) of a user that corresponds to the specific user ID 111.

Note that the counter 55 may be configured or programmed to count the number-of-times-of-use 120 in consideration of a use period 121 (see FIG. 5) in which the receiver 53 has received the reception information 101. In this case, the counter 55 is able to count the number-of-times-of-use 120, based on the user ID 111 and the reading date 112 of the use history table T11. In this case, the counter 55 first specifies the corresponding reading date 112 in a predetermined period (the use period 121) and extracts a row that includes the specified reading date 112. Then, the counter 55 counts rows in which a specific user ID 111 is included among the extracted rows and sets a count number of the counting as the number-of-times-of-use 120 of a user that corresponds to the specific user ID 111.

Furthermore, the counter 55 may be configured or programmed to count the number of times of use for each user and for each of the processes 12 to 16. In this case, the counter 55 is able to count the number of times of use for each of the processes 12 to 16, based on the user ID 111 and the process ID 113 of the use history table T11 illustrated in FIG. 4. In this case, the counter 55 extracts a row that includes the specific user ID 111. Note that the counter 55 may be configured or programmed to perform counting in consideration of a period in which the receiver 53 has received the reception information 101. In this case, the counter 55 further extracts a row that includes the specific user ID 111 from the rows that include the corresponding reading date 112 in the predetermined use period 121. After the counter 55 extracts a row that includes the specific use period 121 and the specific user ID 111 in the above described manner, the counter 55 counts the number of times of use for each of the processes 12 to 16, based on the process ID 113.

In this preferred embodiment, as illustrated in FIG. 5, the number-of-times-of-use 120 includes the number-of-times-of-collection use 122, the number-of-times-of-washing-use 123, the number-of-times-of-assembling-use 124, the number-of-times-of-sterilization-use 125, and the number-of-times-of-storage-use 126. In this case, the number-of-times-of-collection-use 122 is the number of times of use in the collection process 12. The number-of-times-of-washing-use 123 is the number of times of use in the washing process 13. The number-of-times-of-assembling-use 124 is the number of times of use in the assembling process 14. The number-of-times-of-sterilization-use 125 is the number of times of use in the sterilization process 15. The number-of-times-of-storage-use 126 is the number of times of use in the storage process 16.

In this preferred embodiment, the number-of-times-of-use 120 for each user, which has been counted by the counter 55, is stored in the number-of-times-of-use table T12 (see FIG. 5). In FIG. 5, an example of a configuration of the number-of-times-of-use table T12 in a case in which the number of times of use has been counted for each user and for each of the processes 12 to 16. The number-of-times-of-use table T12 is stored in the storage 51 in advance. In this preferred embodiment, the number-of-times-of-use table T12 is stored such that the user ID 111, the use period 121, the number-of-times-of-collection-use 122, the number-of-times-of-washing-use 123, the number-of-times-of-assembling-use 124, the number-of-times-of-sterilization-use 125, and the number-of-times-of-storage-use 126 are associated with one another. Note that some other item than the above described items may be included in the number-of-times-of-use table T12. In this case, the counter 55 counts the number-of-times-of-use 120 for each user and for each of the processes 12 to 16. Thereafter, each of the numbers-of-times-of-use 120 is stored in a corresponding one of the items of the number-of-times-of-use table T12. At this time, the user ID 111 and the use period 121 are also stored in association with the above described items. Note that, in a case in which the number-of-times-of-use 120 is not counted for each of the processes 12 to 16, in the number-of-times-of-use table T12 of FIG. 5, the number-of-times-of-collection-use 122, the number-of-times-of-washing-use 123, the number-of-times-of-assembling-use 124, the number-of-times-of-sterilization-use 125, and the number-of-times-of-storage-use 126 are removed and one item of the number of times of use (the number of times of use which includes those of all of the processes 12 to 16) for each user is included.

The fee calculator 57 calculates a use fee 130 (see FIG. 6) for each user, based on the number-of-times-of-use 120 for each user, which has been counted by the counter 55, and a unit price that has been determined in advance. The fee calculator 57 multiples the number-of-times-of-use 120 for each user, which has been counted by the counter 55, by the unit price that has been determined in advance and thus calculates the use fee 130 (see FIG. 6) for each user. The predetermined unit price is stored in the storage 51 in advance. For example, in a case in which the number of times of use is not counted for each of the processes 12 to 16 by the counter 55 but the number-of-times-of-use 120 which includes those of all of the processes 12 to 16 is counted, only one unit price is set as the above described predetermined unit price.

Note that the unit price may be set in a stepwise manner in accordance with the number-of-times-of-use 120. For example, when the number-of-times-of-use 120 is between a first number of times and a second number of times, which is larger than the first number of times, a first unit price is used and, when the number-of-times-of-use 120 is between the second number of times and a third number of times, which is larger than the second number of times, a second unit price that is lower than the first unit price is used.

For example, as in the number-of-times-of-use table T12 of FIG. 5, in a case in which the number-of-times-of-use 120 for each user and for each of the processes 12 to 16 is counted, the predetermined unit price includes a unit price for collection, a unit price for washing, a unit price for assembling, a unit price for sterilization, and a unit price for storage. In this case, the unit price for collection is a price per use when the medical instrument management system 100 is used in the collection process 12. A collection use fee 132 is calculated based on the number-of-times-of-collection-use 122 and the unit price for collection using a predetermined mathematical expression. In this case, the unit price for collection is multiplied by the number-of-times-of-collection-use 122. The unit price for washing is a price per use when the medical instrument management system 100 is used in the washing process 13. A washing use fee 133 is calculated based on the number-of-times-of-washing-use 123 and the unit price for washing using a predetermined mathematical expression. In this case, the unit price for washing is multiplied by the number-of-times-of-washing-use 123. The unit price for assembling is a price per use when the medical instrument management system 100 is used in the assembling process 14. An assembling use fee 134 is calculated based on the number-of-times-of-assembling-use 124 and the unit price for assembling using a predetermined mathematical expression. In this case, the unit price for assembling is multiplied by the number-of-times-of-assembling-use 124. The unit price for sterilization is a price per use when the medical instrument management system 100 is used in the sterilization process 15. A sterilization use fee 135 is calculated based on the number-of-times-of-sterilization-use 125 and the unit price for sterilization using a predetermined mathematical expression. In this case, the unit price for sterilization is multiplied by the number-of-times-of-sterilization-use 125. The unit price for storage is a price per use when the medical instrument management system 100 is used in the storage process 16. A storage use fee 136 is calculated based on the number-of-times-of-storage-use 126 and the unit price for storage using a predetermined mathematical expression. In this case, the unit price for storage is multiplied by the number-of-times-of-storage-use 126.

Note that the unit price for collection, the unit price for washing, the unit price for assembling, the unit price for sterilization, and the unit price for storage may be the same price and may be different prices. Some of the unit price for collection, the unit price for washing, the unit price for assembling, the unit price for sterilization, and the unit price for storage may be the same price. Each of the above described unit prices may be appropriately set in accordance with the degree of importance of each of the processes 12 to 16. Also, as the unit price for collection, the unit price for washing, the unit price for assembling, the unit price for sterilization, and the unit price for storage, a different price may be set for each user. In this case, the unit price for collection, the unit price for washing, the unit price for assembling, the unit price for sterilization, and the unit price for storage are stored in the storage 51 in advance. In this preferred embodiment, for example, the unit price for collection corresponds to the first unit price of the present invention. The unit price for washing corresponds to the second unit price of the present invention.

In this preferred embodiment, the fee calculator 57 multiplies the number-of-times-of-collection-use 122 of the number-of-times-of-use table T12 by the unit price for collection for each user in the above described manner, thus calculating the collection use fee 132. Similarly, the fee calculator 57 multiplies the number-of-times-of-washing-use 123 by the unit price for washing for each user in the above described manner, thus calculating the washing use fee 133. The fee calculator 57 multiplies the number-of-times-of-assembling-use 124 by the unit price for assembling, thus calculating the assembling use fee 134. Also, the fee calculator 57 multiplies the number-of-times-of-sterilization-use 125 by the unit price for sterilization for each user, thus calculating the sterilization use fee 135. The fee calculator 57 multiplies the number-of-times-of-storage-use 126 by the unit price for storage, thus calculating the storage use fee 136.

In this case, the collection use fee 132, the washing use fee 133, the assembling use fee 134, the sterilization use fee 135, and the storage use fee 136 included in the use fee 130 for each user, which have been calculated by the fee calculator 57, are stored in the use fee table T13 (see FIG. 6). In FIG. 6, an example of a configuration of the use fee table T13 in a case in which the number-of-times-of-use 120 is calculated for each user and for each of the processes 12 to 16 is illustrated. The use fee table T13 is stored in the storage 51 in advance. In this preferred embodiment, the use fee table T13 is stored such that the user ID 111, the use period 121, the collection use fee 132, the washing use fee 133, the assembling use fee 134, the sterilization use fee 135, and the storage use fee 136 are associated with one another. Note that some other item than the above described items may be included in the use fee table T13. For example, an item of a total use fee for each user, which is obtained by adding the five use fees of the use fees 132 to 136, may be included in the use fee table T13. In this case, the fee calculator 57 calculates the use fee 130 for each user and for each of the processes 12 to 16. Thereafter, each of the use fees 130 is stored in a corresponding one of the items of the use fee table T13. Note that, in a case in which the use fee is not calculated for each of the processes 12 to 16, in the use fee table T13 of FIG. 6, each of the items of the use fees 132 to 136 is removed and an item of the use fee (the use fee which includes those of all of the processes 12 to 16) for each user is included.

The notifier 59 notifies the use fee 130 that has been calculated by the fee calculator 57 for each user. Note that there is no particular limitation on a specific configuration in which the notifier 59 notifies each user of the use fee 130. For example, the notifier 59 creates, for each user, an invoice with description of the use fees 132 to 136 and the total use fee obtained by adding up the use fees 132 to 136 in a predetermined format, based on the use fee table T13 of FIG. 6. The notifier 59 may be configured or programmed to transmit the invoice to a printer (not illustrated) communicably connected to the use fee calculation device 50. In this case, the invoice is printed on a paper medium and the invoice of the paper medium is transmitted to the user. Thus, it is possible to notify the user of the use fee 130. Also, the notifier 59 may be configured or programmed to notify the user of the use fee 130 by transmitting the invoice to a predetermined e-mail address. Also, for example, an invoice notification button (not illustrated) may be displayed on the display screen 36 of the terminal 35. In this case, the notifier 59 may be configured or programmed such that the user operates the operation section 37 and presses the invoice notification button, and thus, the notifier 59 causes the display screen 36 to display the use fee 130 thereon. Also, the notifier 59 may be configured or programmed to notify the user of the use fee 130 for each predetermined period. The notifier 59 may be configured or programmed, for example, to cause the display screen 36 to display the use fee 130 thereon in real time.

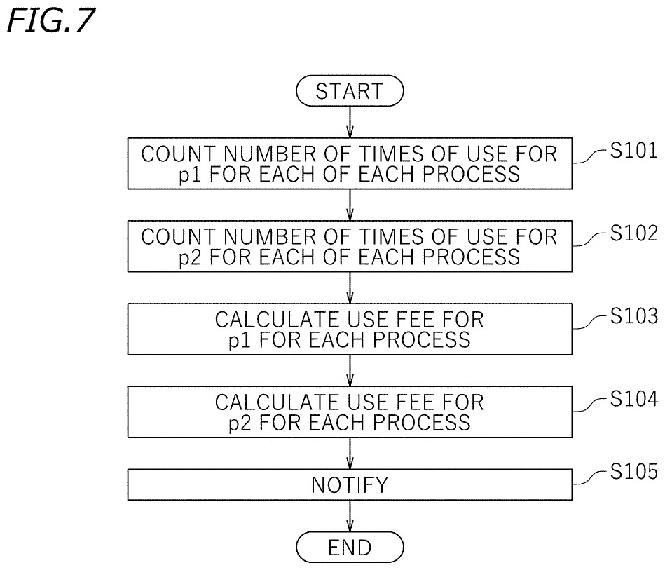

A configuration of the medical instrument management system 100 according to this preferred embodiment has been described above. Next, procedures for calculating the use fee 130 will be described. In this case, as an example, assuming a case in which the operations of the collection process 12, the assembling process 14, the sterilization process 15, and the storage process 16 of the processes 12 to 16 are performed by a user the user ID 111 of which is “p1” and the washing process 13 is performed by a user the user ID 111 of which is “p2”, procedures for calculating the use fee 130 for each of the users p1 and p2 will be described in accordance with a flowchart of FIG. 7. In the following description, the user p1 is the user the user ID 111 of which is “p1”. The user p2 is the user the user ID 111 of which is “p2”.

In this preferred embodiment, before the use fee 130 is calculated, the identification label 6 given to the medical instrument 5 is read by each user using the reading device 30, and management of the medical instrument 5 is performed by the medical instrument management device 40. Therefore, in the use history table T11 stored in the storage 51, as illustrated in FIG. 4, data in which the user ID 111, the reading date 112, and the process ID 113 are associated with one another is stored. Note that, in FIG. 4, a portion of data is illustrated and the other portion of the data is omitted.

In this preferred embodiment, the counter 55 counts the number-of-times-of-use 120 for the user p1, based on the use history table T11, in Step S101. The counter 55 counts the number-of-times-of-use 120 for the user p2, based on the use history table T11, in Step S102. In this case, for each of the users p1 and p2, the number-of-times-of-use 120 of use in the predetermined use period 121 is calculated for each of the processes 12 to 16. The counter 55 extracts, among all of rows of the use history table T11, rows in which the reading date 112 is in “June of 2018 (2018 Jun. 1 to 2018 Jun. 30) which is an example of a range of the predetermined period.

Next, the counter 55 counts, among the extracted rows, rows in which the user is the user p1 and the process ID 113 is “s12” that is the ID of the collection process 12. In this case, the counter 55 sets “50” that is a count number of the counting as the number-of-times-of-collection-use 122 of the user p1 (see FIG. 5). Similarly, the counter 55 counts, among the extracted rows, rows in which the user is the user p1 and the process ID 113 is “s14” that is the ID of the assembling process 14 and sets “60” that is a count number of the counting as the number-of-times-of-assembling-use 124 of the user p1. The counter 55 counts, among the extracted rows, rows in which the user is the user p1 and the process ID 113 is “s15” that is the ID of the sterilization process 15 and sets “50” that is a count number of the counting as the number-of-times-of-sterilization-use 125 of the user p1. The counter 55 counts, among the extracted rows, rows in which the user is the user p1 and the process ID 113 is “s16” that is the ID of the storage process 16 and sets “50” that is a count number of the counting as the number-of-times-of-storage-use 126 of the user p1. Also, the counter 55 counts, among the extracted rows, rows in which the user is the user p2 and the process ID 113 is “s13” that is the ID of the washing process 13 and sets “60” that is a count number of the counting as the number-of-times-of-washing-use 123 of the user p2. Each of the numbers of times of use is the number of times the user has read the identification label 6 of the medical instrument 5 using the reading device 30 in the corresponding process. The number-of-times-of-collection-use 122, the number-of-times-of-assembling-use 124, the number-of-times-of-sterilization-use 125, and the number-of-times-of-storage-use 126 of the user p1 and the number-of-times-of-washing-use 123 of the user p2 are stored in association with the user ID 111 and the use period 121 in the number-of-times-of-use table T12. Note that, in an item of the number of times of use (for example, the number-of-times-of-washing-use 123 of the user p1) which corresponds to a process that is not used by a user, “0” may be put and a so-called null (NULL) character may be put.

Next, the fee calculator 57 calculates the use fee 130 for each of the processes 12 to 16 for the user p1 in Step S103. The fee calculator 57 calculates the use fee 130 for each of the processes 12 to 16 for the user p2 in Step S104. In Step S103, the fee calculator 57 calculates the collection use fee 132 (see FIG. 6) of the user p1 by multiplying “50” of the number-of-times-of-collection-use 122 of the user p1 by “100” that is the unit price for collection, based on the number-of-times-of-use table T12 of FIG. 5. Similarly, the fee calculator 57 calculates the assembling use fee 134 of the user p1 by multiplying “60” of the number-of-times-of-assembling-use 124 of the user p1 by “100” that is the unit price for assembling. The fee calculator 57 calculates the sterilization use fee 135 of the user p1 by multiplying “50” of the number-of-times-of-sterilization-use 125 of the user p1 by “150” that is the unit price for sterilization. The fee calculator 57 calculates the storage use fee 136 of the user p1 by multiplying “50” of the number-of-times-of-storage-use 126 of the user p1 by “100” that is the unit price for storage. In Step S104, the fee calculator 57 calculates the washing use fee 133 (see FIG. 6) of the user p2 by multiplying “60” of the number-of-times-of-washing-use 123 of the user p2 by “150” that is the unit price for washing. The collection use fee 132, the assembling use fee 134, the sterilization use fee 135, and the storage use fee 136 of the user p1 and the washing use fee 133 of the user p2 that have been calculated are stored in association with the user ID 111 and the use period 121 in the use fee table T13. Note that, in an item of the use fee (for example, the washing use fee 133 of the user p1) which corresponds to a process that is not used by the user, “0” may be put and a so-called null (NULL) character may be put.

Next, in Step S105, the notifier 59 notifies the use fee 130 by issuing an invoice for each of the users p1 and p2. In this case, for example, the notifier 59 creates an invoice for the user p1 with description of the collection use fee 132, the assembling use fee 134, the sterilization use fee 135, the storage use fee 136, and a total use fee of the user p1 in a predetermined format, based on the use fee table T13. Similarly, the notifier 59 creates an invoice for the user p2 with description of the washing use fee 133 (in this case, the total use fee) of the user p2, based on the use fee table T13. Then, the notifier 59 transmits the invoice for the user p1 to the terminal 35 (see FIG. 1) of the user p1 and transmits the invoice for the user p2 to the terminal 35 of the user p2. Thus, the use fee 130 for each of the user p1 and the user p2 in the predetermined use period is able to be notified. Note that, as described above, the user may be not only a person but also an organization, such as an enterprise, a group, a hospital, or the like, a section of the organization, a process, or the like. For example, in a case in which the use fee 130 has been calculated for each organization, the notifier 59 is configured or programmed to notify the use fee 130 for each organization, such as an enterprise, a group, a hospital, or the like. For example, in a case in which the use fee 130 has been calculated for each section of an organization or for each process, the notifier 59 is configured or programmed to notify the use fee 130 for each section or for each process.

As described above, in this preferred embodiment, each time a user reads the identification label 6 of the medical instrument 5 using the reading device 30, the receiver 53 receives the reception information 101. Therefore, the use fee 130 in accordance with the number-of-times-of-use 120 for the user can be calculated by counting the number-of-times-of-use 120 that is the number of times the reception information 101 has been received for each user and multiplying the number-of-times-of-use 120 by a predetermined unit price. Accordingly, for a user the number-of-times-of-use 120 of which is small, a relatively reasonable use fee 130 can be charged and, for a user the number-of-times-of-use 120 of which is large, a relatively expensive use fee 130 can be charged. Therefore, the use fee 130 in accordance with the number-of-times-of-use 120 of each user can be charged to the user. Therefore, a proper use fee 130 for the user can be calculated.

For example, for a small- or medium-scaled hospital, it is presumably difficult to introduce a medical instrument management system for which an expensive fee is charged. Therefore, for a small- or medium-scaled hospital, a barrier to introduction of a medical instrument management system might be high. However, in some cases, a small- or medium-scaled hospital owns fewer medical instruments 5 than a large-scaled hospital. In this case, the number-of-times-of-use 120 of the medical instrument management system 100 is relatively small. Therefore, as in this preferred embodiment, by setting the use fee 130 in accordance with the number-of-times-of-use 120, it is possible to facilitate introduction of the medical instrument management system 100 by a small- or medium-scaled hospital, as compared to a conventional medical management system, and also to lower a barrier to introduction of the medical instrument management system 100.

Also, in this preferred embodiment, when a plurality of reading devices 30 read the identification label 6, each of the plurality of reading device 30 receives the reception information 101. In this case, the medical instrument management system 100 is a client-server system. Therefore, for example, even in a case in which the reading devices 30 are installed in remote locations, the reception information 101 can be put together in one place. Accordingly, the use fee 130 for each user can be calculated in a place in which the reception information 101 has been received. Thus, the use fee 130 can be calculated without a visit of a provider to the places in which the reading devices 30 are installed. Therefore, for the provider of the medical instrument management device 40, an operation time related to the use fee 130 is able to be reduced.

In this preferred embodiment, as illustrated in FIG. 4, the read information 102 of the reception information 101 includes the reading date 112. The counter 55 sets the number of times the reception information 101 has been received in a predetermined period (the use period 121 (see FIG. 6)) as the number-of-times-of-use 120 for each user, based on the reading date 112. Thus, the use fee 130 in the use period 121 that has been designated is able to be calculated for each user. Accordingly, for example, the use fee 130 for each user is able to be easily calculated on a monthly basis.

In this preferred embodiment, for each user, the notifier 59 notifies the use fee 130 that has been calculated by the fee calculator 57. Thus, for each user, the use fee 130 for the user is able to be readily notified.

In this preferred embodiment, in each of the predetermined processes 12 to 16 in which predetermined operations are performed on the medical instrument 5, the identification label 6 given to the medical instrument 5 is read before the operation is preformed, and thus, the receiver 53 receives the reception information 101. Thus, in each of the processes 12 to 16, the reception information 101 is able to be received before the operation. Therefore, the number-of-times-of-use 120 is able to be more accurately counted for each user.

In this preferred embodiment, the use fee 130 is calculated for each of the processes 12 to 16 by counting the number-of-times-of-use 120 for each of the processes 12 to 16 and multiplying a count number of the counting by a unit price that has been set for each of the processes 12 to 16. For example, in the collection process 12, the number-of-times-of-collection-use 122 is counted by counting the reception information 101 when the reception information 101 has been read by the reading device 30, and the number-of-times-of-collection-use 122 is multiplied by a unit price for collection. Thus, a unit price for use in each of the processes 12 to 16 can be set for each of the processes 12 to in accordance with the degree of importance of each of the processes 12 to 16 by calculating the use fee 130 for each of the processes 12 to 16.

### Second Preferred Embodiment

The medical instrument management system 100 according to the first preferred embodiment has been described above. Next, a medical instrument management system 200 according to a second preferred embodiment will be described. In the first preferred embodiment, the number-of-times-of-use 120, that is, the number of times the medical instrument management system 100 has been used in each of processes 12 to 16, is calculated and the use fee 130 is calculated by multiplying the number-of-times-of-use 120 that has been calculated by a predetermined unit price. Then, the use fee 130 described above is charged to a user. However, the medical instrument management system 200 according to the second preferred embodiment is configured or programmed such that a manual use fee is charged to the user in accordance with the number of times an electronic manual of the medical instrument management system 200 has been browsed as well as the use fee 130 in accordance with the number-of-times-of-use 120, that is, the number of times of use in each of the processes 12 to 16, which has been described above.

In the following description, a component that is similar to a corresponding one of the components that have been already described above is denoted by the same reference character as that of the corresponding component, and description thereof will be appropriately omitted.

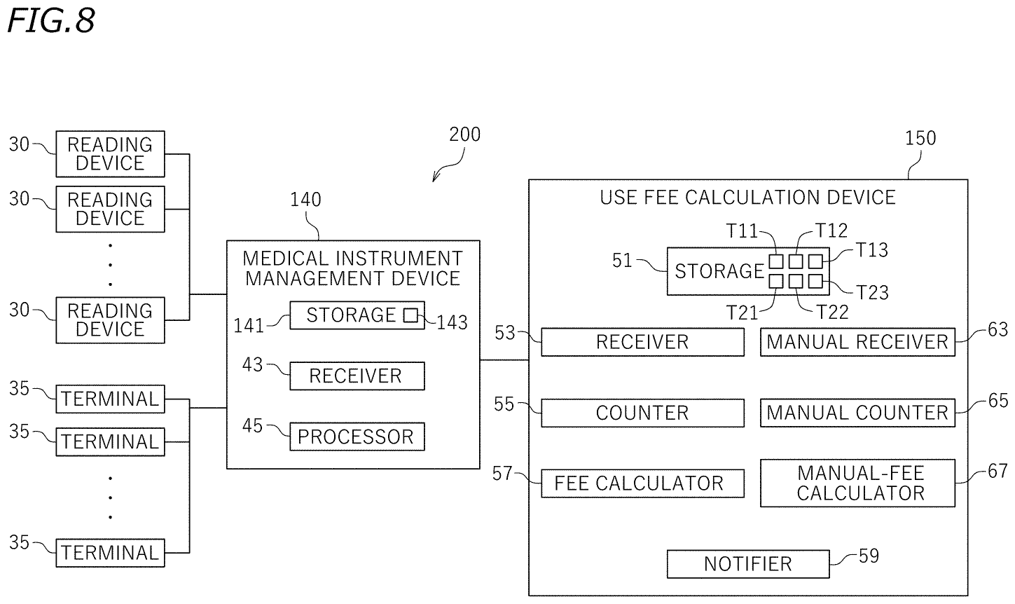

FIG. 8 is a block diagram of the medical instrument management system 200 according to the second preferred embodiment. In this preferred embodiment, as illustrated in FIG. 8, similar to the first preferred embodiment, the medical instrument management system 200 includes at least one reading device 30, at least one terminal 35, a medical instrument management device 140, and a use fee calculation device 150. In this case, the reading device 30 has the same configuration as that of the first preferred embodiment, and therefore, description thereof will be omitted in this preferred embodiment.

The medical instrument management device 140 includes a storage 141, and a receiver 43 and a processor 45 that are similar to those of the first preferred embodiment. In the storage 141, an electronic manual 143 is stored. The electronic manual 143 is a manual in which operation procedures in the processes 12 to 16 in each medical instrument 5 are described. For example, the electronic manual 143 is a manual in which, for one medical instrument 5, procedures for disassembling the medical instrument 5 in the collection process 12, procedures for washing the medical instrument 5 in the washing process 13, procedures for assembling the medical instrument 5 in the assembling process 14, or the like are described. The electronic manual 143 may not be prepared for all of the medical instruments 5. The electronic manual 143 may be prepared for, for example, only the medical instrument 5 that is formed of a plurality of components and has a complicated configuration. For example, in a case in which the medical instrument 5 is forceps, the forceps are not formed of a plurality of components and has a simple configuration. Therefore, the electronic manual 143 for the forceps may not be prepared. The electronic manual 143 is, for example, in a portable document format (PDF).

In this preferred embodiment, the terminal 35 is communicably connected to the medical instrument management device 140. It is possible to access the electronic manual 143 in the medical instrument management device 140 by operating the terminal 35. Also, it is possible to display the electronic manual 143 on a display screen 36 (see FIG. 2) of the terminal 35 by operating the electronic manual 143. The user can browse the electronic manual 143 via the display screen 36 (see FIG. 2) of the terminal 35.

In this preferred embodiment, similar to the use fee calculation device 50 according to the first preferred embodiment, the use fee calculation device 150 includes a storage 51, a receiver 53, a counter 55, a fee calculator 57, and a notifier 59. In this preferred embodiment, the use fee calculation device 150 further includes a manual receiver 63, a manual counter 65, and a manual-fee calculator 67. The manual receiver 63, the manual counter 65, and the manual-fee calculator 67 are used to calculate a manual use fee in accordance with use of the electronic manual 143. Each of the above described components may be formed of a software and may be formed of a hardware. Also, in each of the above described components may be realized by one or more processors and may be incorporated in a circuit.

Next, an example of procedures for calculating the manual use fee in accordance with use of the electronic manual 143 will be described. Specifically, an example of procedures for calculating the manual use fee for each of manual users p1 and p2 that are users of the electronic manual 143 will be described in accordance with a flowchart of FIG. 12. The manual users will be hereinafter occasionally referred to as users.

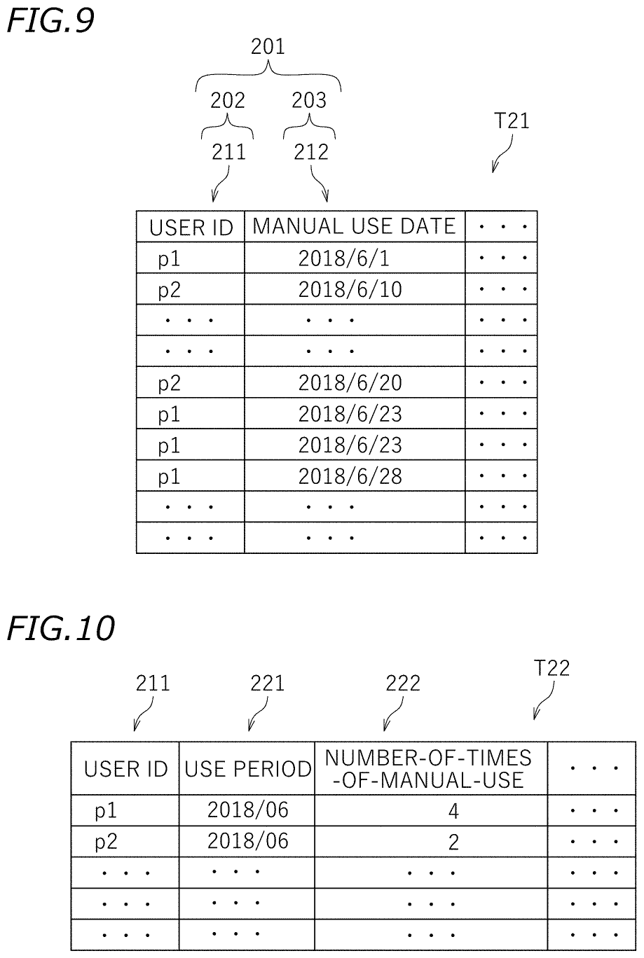

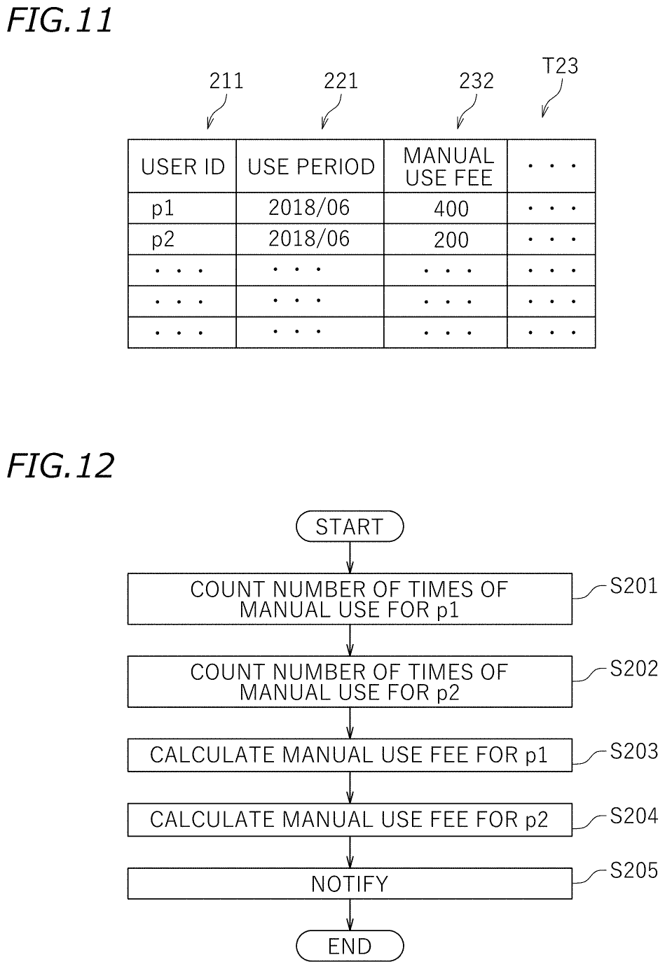

FIG. 9 is a table illustrating an example of a configuration of a manual use history table T21. FIG. 10 is a table illustrating an example of a configuration of a number-of-times-of-manual-use table T22. FIG. 11 is a table illustrating an example of a configuration of a manual use fee table T23. In this preferred embodiment, in the storage 51, the manual use history table T21, the number-of-times-of-manual-use table T22, and the manual use fee table T23 are stored.

In this case, before control is performed in accordance with the flowchart of FIG. 12, the manual receiver 63 receives manual reception information 201 (see FIG. 9). The manual reception information 201 herein is information that is transmitted to the manual receiver 63 when the electronic manual 143 is accessed. In this preferred embodiment, the manual reception information 201 includes access information 203 and manual user information 202. For example, in this preferred embodiment, a display button (not illustrated) that causes the electronic manual 143 to be displayed is displayed on the display screen 36 of the terminal 35. The display button is linked to the electronic manual 143. The access information 203 is, for example, information when the user has accessed the electronic manual 143 by operating the terminal 35. As used herein, an “access” is a state in which, for example, the above described display button that is linked to the electronic manual 143 is clicked via a mouse and the electronic manual 143 is displayed on the display screen 36 (see FIG. 2) of the terminal 35. In this preferred embodiment, the access information 203 includes a manual use date 212. The manual use date 212 is a date on which the user has accessed the electronic manual 143 via the terminal 35 or the like. For example, when the electronic manual 143 is accessed, the medical instrument management device 140 acquires a date on which the user has accessed the electronic manual 143 and sets the date as the manual use date 212. Note that the manual use date 212 may include, in addition to the date, a time at which the user has accessed the electronic manual 143.

The manual user information 202 is information that specifies a user who has accessed the electronic manual 143. The manual user information 202 is recorded in association with the access information 203. The manual user information 202 includes, for example, a user ID 211. In this preferred embodiment, the user ID 211 is stored in the terminal 35 in advance. When a user accesses the electronic manual 143 of the medical instrument management device 140 from the terminal 35, the user ID 211 that has been stored in the terminal 35 in advance is transmitted to the medical instrument management device 140. The medical instrument management device 140 transmits the manual use date 212 with the user ID 211 that has been received to the manual receiver 63. The manual receiver 63 causes the storage 51 to store the user ID 211 and the manual use date 212 in association with one another therein.

In this preferred embodiment, when the manual reception information 201 is received by the manual receiver 63, the manual reception information 201 is stored in the manual use history table T21 of FIG. 9. The manual use history table T21 is a table in which a history in which the user has accessed the electronic manual 143 is recorded. The manual use history table T21 is stored in the storage 51 in advance. In this preferred embodiment, in the manual use history table T21, items of the user ID 211 and the manual use date 212 are stored in association with one another. Note that some other item than the above described two items may be included in the manual use history table T21. Although not illustrated, the process ID 113 illustrated in FIG. 4 may be included in the manual use history table T21. Thus, it is possible to manage a use history of the electronic manual 143 for each of the processes 12 to 16.

In this preferred embodiment, before a manual use fee is calculated, the identification label 6 given to the medical instrument 5 is read by each user using the reading device 30 and management of the medical instrument 5 is performed by the medical instrument management device 40. Therefore, in the manual use history table T21 stored in the storage 51, as illustrated in FIG. 9, data in which the user ID 211 and the manual use date 212 are associated with one another is stored. Note that, in FIG. 9, a portion of the data is illustrated and the other portion of the data is omitted.

The manual counter 65 counts, based on the manual reception information 201, the number-of-times-of-manual-use 222 (see FIG. 10) which is the number of pieces of the access information 203 that have been received by the manual receiver 63 for each manual user. In this case, the manual counter 65 counts the number-of-times-of-manual-use 222 (see FIG. 10) for the manual user p1 in Step S201 of FIG. 12. The manual counter 65 counts the number-of-times-of-manual-use 222 for the manual user p2 in Step S202 of FIG. 12. In this preferred embodiment, the manual counter 65 counts, based on the manual reception information 201 which has been received by the manual receiver 63, the number-of-times-of-manual-use 222 for each of the manual users p1 and p2. In this case, the number-of-times-of-manual-use 222 is the number of times the manual receiver 63 has received the manual reception information 201 for each of the manual users p1 and p2. In this case, the manual counter 65 counts, based on the manual use history table T21, the number-of-times-of-manual-use 222 which is the number of times the manual receiver 63 has received the manual reception information 201 for each of the manual users p1 and p2. The manual counter 65 counts rows in which a specific user ID 211 is included in the manual use history table T21 of FIG. 9. Then, the number of the rows, which has been counted, is set as the number-of-times-of-manual-use 222 for the manual user that corresponds to the specific user ID 211.

Note that the manual counter 65 may be configured or programmed to count the number-of-times-of-manual-use 222 in consideration of a period in which the manual receiver 63 has received the manual reception information 201. In this case, the manual counter 65 is able to count, based on the user ID 211 and the manual use date 212 of the manual use history table T21, the number-of-times-of-manual-use 222. In this case, the manual counter 65 first specifies the corresponding manual use date 212 in a predetermined period (for example, a user period 221 (see FIG. 10)) and extracts a row that includes the specified manual use date 212. Then, the manual counter 65 counts a row in which the specific user ID 211 is included among the extracted rows and sets a count number of the counting, as the number-of-times-of-manual-use 222 for one of the manual users p1 and p2 which corresponds to the specific user ID 211.

In this preferred embodiment, the number-of-times-of-manual-use 222 for each of the manual users p1 and p2, which has been counted by the manual counter 65, is stored in the number-of-times-of-manual-use table T22 (see FIG. 10). The number-of-times-of-manual-use table T22 is stored in the storage 51 in advance. In this case, the number-of-times-of-manual-use table T22 is stored such that the user ID 211, the user period 221, and the number-of-times-of-manual-use 222 are associated with one another. Note that some other item than the above described items may be included in the number-of-times-of-manual-use table T22. The number-of-times-of-manual-use table T22 may have a similar configuration to, for example, that of the number-of-times-of-use table T12 illustrated in FIG. 5. That is, the number-of-times-of-manual-use 222 may be broken up into respective numbers-of-times-of-manual-use of the processes 12 to 16. In this case, the number of times of collection use, the number of times of washing use, the number of times of assembling use, the number of times of sterilization use, and the number of times of storage use are included in the number-of-times-of-manual-use table T22. Thus, the number of times the electronic manual 143 has been used for each of the processes 12 to 16 is able to be counted. The manual counter 65 causes, after counting the number-of-times-of-manual-use 222 for each of the manual users p1 and p2, the user ID 211, the user period 221, and the number-of-times-of-manual-use 222 of each of the manual users p1 and p2 to be stored in association with one another in the number-of-times-of-manual-use table T22.

Note that, in an example of FIG. 10, in Step S201 of FIG. 12, the manual counter 65 extracts a row in which the user ID 211 is the manual user p1 and the user period 221 is the manual use date 212 that corresponds to “June, 2018” among the rows of the manual use history table T21 (see FIG. 9) and counts the rows that have been extracted. Then, the manual counter 65 sets a count number of the counting as the number-of-times-of-manual-use 222 for the manual user p1. In this case, the number-of-times-of-manual-use 222 for the manual user p1 is “4”. In Step S202 of FIG. 12, the manual counter 65 extracts a row in which the user ID 211 is the manual user p2 and the user period 221 is the manual use date 212 that corresponds to “June, 2018” among the rows of the manual use history table T21 (see FIG. 9) and counts the rows that have been extracted. Then, the manual counter 65 sets a count number of the counting as the number-of-times-of-manual-use 222 for the manual user p2. In this case, the number-of-times-of-manual-use 222 for the manual user p2 is “2”.

Next, the manual-fee calculator 67 calculates, based on the number-of-times-of-manual-use 222 for each manual user, which has been counted by the manual counter 65, and a manual unit price that has been set in advance, a use fee for each manual user. In this case, in Step S203, the manual-fee calculator 67 calculates a manual use fee 232 (see FIG. 11) for the user p1. Then, in Step S204, the manual-fee calculator 67 calculates the manual use fee 232 for the user p2. In this case, the manual-fee calculator 67 calculates the use fee 232 for each of the manual users p1 and p2 by multiplying the number-of-times-of-manual-use 222 for each of the manual users p1 and p2, which has been counted by the manual counter 65, by the predetermined manual unit price that has been set in advance. The manual unit price is stored in the storage 51 in advance. The manual unit price may be a fixed unit price for the plurality of users p1 and p2 and may be different values for the plurality of manual users p1 and p2 or some of the manual users.

The use fee 232 for each of the manual users p1 and p2, which has been calculated by the manual-fee calculator 67, is stored in the manual use fee table T23 (see FIG. 11). The manual use fee table T23 is stored such that the user ID 211, the user period 221, and the manual use fee 232 are associated with one another. Note that some other item than the above described items may be included in the manual use fee table T23. The manual use fee table T23 may have a similar configuration to, for example, that of the use fee table T13 illustrated in FIG. 6. That is, the manual use fee 232 may be broken up into respective manual use fees of the processes 12 to 16. In this case, a collection use fee, a washing use fee, an assembling use fee, a sterilization use fee, and a storage use fee are included in the manual use fee table T23. Thus, the use fee of the electronic manual 143 that has been used for each of the processes 12 to 16 is able to be calculated. Note that the manual unit price for each of the collection use fee, the washing use fee, the assembling use fee, the sterilization use fee, and the storage use fee when the collection use fee, the washing use fee, the assembling use fee, the sterilization use fee, and the storage use fee are calculated may be the same unit price and may be different unit prices for some of the fees. The unit price that is used when the collection use fee, the washing use fee, the assembling use fee, the sterilization use fee, and the storage use fee are calculated may be set for each of the processes 12 to 16.

Note that, in an example of FIG. 11, in Step S203 of FIG. 12, the manual-fee calculator 67 calculates the manual use fee 232 for the user p1 by multiplying “4” that is the number-of-times-of-manual-use 222 for the user p1 by “100” that is the manual unit price. In Step S204 of FIG. 12, the manual-fee calculator 67 calculates the manual use fee 232 for the user p2 by multiplying “2” that is the number-of-times-of-manual-use 222 for the user p2 by “100” that is the manual unit price for the user p2.

In the above described manner, after the manual use fee 232 is calculated, in Step S205, for each of the manual users p1 and p2, the notifier 59 notifies the manual use fee 232. Note that, similar to the first preferred embodiment, as a notification method performed by the notifier 59, the manual use fee 232 is described in an invoice in a predetermined format. Then, the notifier 59 may be configured or programmed to cause a printer to print the invoice and may be configured or programmed to transmit the invoice to the terminal 35 of each of the manual users p1 and p2. Also, the notifier 59 may be configured or programmed, for example, such that a user operates the operation section 37 to press an invoice notification button (not illustrated) displayed on the display screen 36 of the terminal 35 and thus the notifier 59 causes the manual use fee 232 to be displayed on the display screen 36. Also, the notifier 59 may be configured or programmed to, for example, cause the use fee 130 to be displayed on the display screen 36 in real time. Also, the notifier 59 may be configured or programmed to notify, in a case in which the user of the first preferred embodiment and the manual user of this preferred embodiment are the same, the user of the manual use fee 232 with the use fee for each of the processes 12 to 16.

As described above, in this preferred embodiment, in addition to the use fee in accordance with the number of times the identification label 6 of the medical instrument 5 has been read by the reading device 30, the manual use fee 232 is set in accordance with the number of times the electronic manual 143 has been accessed. In this case, the use fee 232 of the electronic manual 143 may be the meter-rate use fee 232. Therefore, the use fee 232 in accordance with the number of times of accesses to the electronic manual 143 is able to be charged. Accordingly, the proper manual use fee 232 for a user is able to be calculated.

In each of the above described preferred embodiments, the receiver 53 of a corresponding one of the use fee calculation devices 50 and 150 receives the reception information 101 from a corresponding one of the medical instrument management devices 40 and 140. However, the receiver 53 may be configured or programmed to directly receive the reception information 101 from the reading device 30 when the reading device 30 reads the identification label 6 of the medical instrument 5. In this case, the reading date 112 of the reception information 101 may be omitted. In this case, each of the use fee calculation devices 50 and 150 may be configured or programmed to acquire a date on which the receiver 53 has received the reception information 101.

In each of the above described preferred embodiments, the receiver 53 receives the reception information 101 at a timing at which the identification label 6 of the medical instrument 5 has been read by the reading device 30. However, the receiver 53 may be configured or programmed to, for example, collectively receive the reception information 101 in a predetermined period on a predetermined date. The predetermined date in this case is, for example, a date on which the use fee 120 for each user is calculated.

In each of the above described preferred embodiments, the reading device 30 is communicably connected to a corresponding one of the medical instrument management devices 40 and 140 and is indirectly and communicably connected to a corresponding one of the use fee calculation devices 50 and 150 via the corresponding one of the medical instrument management devices 40 and 140. However, the reading device 30 may be communicably connected to the terminal 35 and is indirectly connected to the corresponding one of the use fee calculation devices 50 and 150 via the terminal 35 and the corresponding one of the medical instrument management devices 40 and 140. Also, the reading device 30 may be communicably connected directly to the corresponding one of the use fee calculation devices 50 and 150.

Note that, in each of the above described preferred embodiments, a computer program may be read by a computer, and thus, each of the components of a corresponding one of the medical instrument management devices 40 and 140 and the components of a corresponding one of the use fee calculation devices 50 and 150 may be realized by the computer. Preferred embodiments of the present invention include a computer program that causes a computer to function as each of the components of the medical instrument management devices 40 and 140 and the components of the use fee calculation devices 50 and 150. Also, preferred embodiments of the present invention include a computer-readable recording medium in which the computer program is recorded.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A use fee calculation device for a medical instrument management device, the use fee calculation device comprising:
a receiver that is directly or indirectly connected to at least one reading device and receives read information when an identification label provided in a medical instrument has been read by the reading device and user information that is recorded in association with the read information and specifies a user who uses the reading device;
a counter that counts, based on the read information and the user information that have been received by the receiver, a number of times of use, which is a number of pieces of the read information that have been received by the receiver, for each of the users; and
a fee calculator that calculates, based on the number of times of use for each of the users, which has been counted by the counter, and a unit price that has been set in advance, a use fee for each of the users.

2. The use fee calculation device for a medical instrument management device according to claim 1, wherein the fee calculator calculates the use fee by multiplying the number of times of use for each of the users, which has been counted by the counter, by the unit price.

3. The use fee calculation device for a medical instrument management device according to claim 1, wherein a reading date when the identification label given to the medical instrument has been read by the reading device is included in the read information.

4. The use fee calculation device for a medical instrument management device according to claim 3, wherein the counter counts, based on the reading date, the number of times of use in a predetermined period for each of the users.

5. The use fee calculation device for a medical instrument management device according to claim 1, wherein the receiver receives, in a predetermined process in which a predetermined operation is performed on the medical instrument, the read information and the user information when the identification label given to the medical instrument has been read before the operation is performed.

6. The use fee calculation device for a medical instrument management device according to claim 5, wherein
the predetermined process includes a first process and a second process;
the read information includes process information that corresponds to the predetermined process when the identification label given to the medical instrument has been read;
the counter counts the number of times of use for each of the users and for each of the predetermined processes; and
the fee calculator calculates, based on the number of times of use for the first process and a first unit price that has been set in advance, the use fee for the first process and calculates, based on the number of times of use for the second process and a second unit price that has been set in advance, the use fee for the second process.

7. The use fee calculation device for a medical instrument management device according to claim 5, wherein
the predetermined process includes a collection process related to an operation of collecting the medical instrument;
the receiver receives the read information and the user information when the identification label given to the medical instrument has been read before the collection process is performed;
the counter counts, for each of the users, the number of times of use for the collection process; and
the fee calculator calculates, based on the number of times of use for the collection process and a unit price for collection, which has been set in advance, the use fee in the collection process.

8. The use fee calculation device for a medical instrument management device according to claim 5, wherein
the predetermined process includes a washing process related to an operation of washing the medical instrument;
the receiver receives the read information and the user information when the identification label given to the medical instrument has been read before the washing process is performed;
the counter counts, for each of the users, the number of times of use for the washing process; and
the fee calculator calculates, based on the number of times of use for the washing process and a unit price for washing, which has been set in advance, the use fee in the washing process.

9. The use fee calculation device for a medical instrument management device according to claim 5, wherein
the predetermined process includes an assembling process related to an operation of assembling the medical instrument;
the receiver receives the read information and the user information when the identification label given to the medical instrument has been read before the assembling process is performed;
the counter counts, for each of the users, the number of times of use for the assembling process; and
the fee calculator calculates, based on the number of times of use for the assembling process and a unit price for assembling, which has been set in advance, the use fee in the assembling process.

10. The use fee calculation device for a medical instrument management device according to claim 5, wherein
the predetermined process includes a sterilization process related to an operation of sterilizing the medical instrument;
the receiver receives the read information and the user information when the identification label given to the medical instrument has been read before the sterilization process is performed;
the counter counts, for each of the users, the number of times of use for the sterilization process; and
the fee calculator calculates, based on the number of times of use for the sterilization process and a unit price for sterilization, which has been set in advance, the use fee in the sterilization process.

11. The use fee calculation device for a medical instrument management device according to claim 5, wherein
the predetermined process includes a storage process related to an operation of storing the medical instrument;
the receiver receives the read information and the user information when the identification label given to the medical instrument has been read before the storage process is performed;
the counter counts, for each of the users, the number of times of use for the storage process; and
the fee calculator calculates, based on the number of times of use for the storage process and a unit price for storing, which has been set in advance, the use fee in the storage process.

12. The use fee calculation device for a medical instrument management device according to claim 1, further comprising:
a notifier that notifies the use fee that has been calculated by the fee calculator for each of the users.

13. The use fee calculation device for a medical instrument management device according to claim 1, further comprising:
a manual receiver that receives, in the medical instrument management device that is a medical instrument management device that manages the medical instrument and includes a storage in which an electronic manual is stored, manual reception information in which access information when the electronic manual has been accessed and manual user information that specifies a manual user who uses the electronic manual are associated with one another;
a manual counter that counts, based on the manual reception information, the number of times of manual use, which is the number of pieces of the access information that have been received by the manual receiver, for each of the manual users; and
a manual fee calculator that calculates, based on the number of times of manual use for each of the manual users, which has been counted by the manual counter, and a manual unit price that has been set in advance, a use fee for each of the manual users.

14. A medical instrument management system comprising:
the use fee calculation device according to claim 1; and
a medical instrument management device that is communicably connected to the use fee calculation device and manages the medical instrument.
